(19) United States
(12) Patent Application Publication
(10) Pub. No.: US 2010/0022770 A1
(43) Pub. Date: Jan. 28, 2010

(54) PROCESS FOR THE MANUFACTURING OF BETA MIMETICS

(75) Inventors: Juan M. Rodriguez Dehli, Mainz (DE); Robert Hagenkoetter, Huenstetten (DE); Michael Schul, Lahnstein (DE); Christian Stange, Ingelheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/522,008

(22) PCT Filed: Jan. 24, 2008
(86) PCT No.: PCT/EP08/50800

§ 371 Date: Sep. 11, 2009

(30) Foreign Application Priority Data
Jan. 25, 2007 (EP) 07101130.8

(51) Int. Cl. C07D 265/36

(52) U.S. Cl. 544/105

(57) ABSTRACT

The invention relates to a method for producing betamimetics of formula (1), wherein in which n represents 1 or 2; R¹ represents hydrogen, halogen, C₁₋₄-alkyl or O—C₁₋₄-alkyl; R² represents hydrogen, halogen, C₁₋₄-alkyl or O—C₁₋₄-alkyl and; R³ represents hydrogen, C₁₋₄-alkyl, OH, halogen, O—C₁₋₄-alkyl, O—C₁₋₄-alkylene-COOH or O—C₁₋₄-alkyl-lene-COO—C₁₋₄-alkyl.

The present invention relates to a process for preparing betamimetics of formula 1,

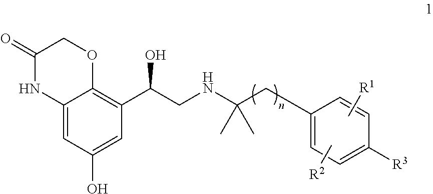

wherein
n denotes 1 or 2;
R¹ denotes hydrogen, halogen, C₁₋₄-alkyl or O—C₁₋₄-alkyl;
R² denotes hydrogen, halogen, C₁₋₄-alkyl or O—C₁₋₄-alkyl;
R³ denotes hydrogen, C₁₋₄-alkyl, OH, halogen, O—C₁₋₄-alkyl, O—C₁₋₄-alkylene-COOH, O—C₁₋₄-alkylene-COO—C₁₋₄-alkyl.

### BACKGROUND TO THE INVENTION

Betamimetics (β-adrenergic substances) are known from the prior art. In this respect reference may be made for example to the disclosure of U.S. Pat. No. 4,460,581 which proposes betamimetics for the treatment of a wide range of ailments.

For drug treatment of diseases it is often desirable to prepare medicaments with a longer duration of activity. As a rule, this ensures that the concentration of the active substance in the body needed to achieve the therapeutic effect is maintained for a longer period without the need to re-administer the drug at frequent intervals. Moreover, giving an active substance at longer time intervals contributes to the well-being of the patient to a high degree. It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to provide a process for preparing betamimetics which on the one hand provide a therapeutic benefit in the treatment of COPD or asthma and are also characterised by a longer duration of activity and can thus be used to prepare pharmaceutical compositions with a longer duration of activity. A particular aim of the invention is to prepare betamimetics which, by virtue of their long-lasting effect, can be used to prepare a drug for the treatment of COPD or asthma for administration once a day. In addition to these aims, a further objective of the invention is to provide such betamimetics which are not only exceptionally potent but are also characterised by a high degree of selectivity with respect to the β₂-adreno-receptor.

### DETAILED DESCRIPTION OF THE INVENTION

The present invention relates to a process for preparing a compound of formula 1,

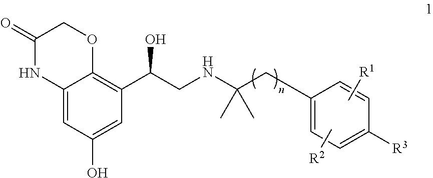

wherein
n denotes 1 or 2;
R¹ denotes hydrogen, halogen, C₁₋₄-alkyl or O—C₁₋₄-alkyl;
R² denotes hydrogen, halogen, C₁₋₄-alkyl or O—C₁₋₄-alkyl;
R³ denotes hydrogen, C₁₋₄-alkyl, OH, halogen, O—C₁₋₄-alkyl, O—C₁₋₄-alkylene-COOH, O—C₁₋₄-alkylene-COO—C₁₋₄-alkyl;
characterised in that a compound of formula 1a,

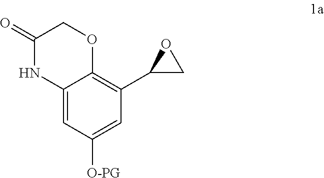

wherein PG denotes a protective group, is reacted with a compound of formula 1b,

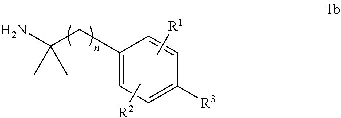

wherein R¹, R², R³ and n have the meanings given above, in an organic solvent, to form a compound of formula 1c,

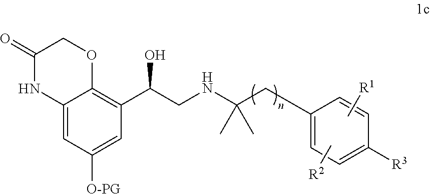

wherein R¹, R², R³, n and PG have the meanings given above, and the compound of formula 1 is obtained therefrom by cleaving the protective group PG.

Preferably the above process is used for preparing compounds of formula 1, wherein

n denotes 1 or 2;
R¹ denotes hydrogen, halogen or C₁₋₄-alkyl;
R² denotes hydrogen, halogen or C₁₋₄-alkyl;
R³ denotes hydrogen, C₁₋₄-alkyl, OH, halogen, O—C₁₋₄-alkyl, O—C₁₋₄-alkylene-COOH or O—C₁₋₄-alkylene-COO—C₁₋₄-alkyl.

Preferably the above process is used for preparing compounds of formula 1, wherein

n denotes 1 or 2;
R¹ denotes hydrogen, fluorine, chlorine, methyl or ethyl;
R² denotes hydrogen, fluorine, chlorine, methyl or ethyl;
R³ denotes hydrogen, C₁₋₄-alkyl, OH, fluorine, chlorine, bromine, O—C₁₋₄-alkyl, O—C₁₋₄-alkylene-COOH, O—C₁₋₄-alkylene-COO—C₁₋₄-alkyl.

Preferably the above process is used for preparing compounds of formula 1, wherein

n denotes 1 or 2;
R¹ denotes hydrogen, methyl or ethyl;
R² denotes hydrogen, methyl or ethyl;
R³ denotes hydrogen, methyl, ethyl, OH, methoxy, ethoxy, O—CH₂—COOH, O—CH₂—COO-methyl or O—CH₂—COO-ethyl.

Preferably the above process is used for preparing compounds of formula 1, wherein

n denotes 1 or 2;
R¹ denotes hydrogen or methyl;
R² denotes hydrogen or methyl;
R³ denotes hydrogen, methyl, OH, methoxy, O—CH₂—COOH or O—CH₂—COO-ethyl.

In the process according to the invention a compound of formula 1a is reacted in toluene with a compound of formula 1b. The compound of formula 1b may be in the form of a base. For this, the corresponding salt (e.g. hydrochloride) is extracted with an excess of a strong base (sodium hydroxide solution, potassium hydroxide solution, etc.) in an organic solvent, preferably toluene.

Preferably at least stoichiometric amounts of compound 1b are used according to the invention, based on the compound 1a used. If desired compound 1b may also be used in an excess, for example up to 3 equivalents, preferably up to 2.5 equivalents, particularly preferably about 1 to 2, optionally 1 to 1.5 equivalents, based on the compound 1a used.

The reaction is preferably carried out at elevated temperature, preferably at a temperature of over 40° C., particularly preferably at a temperature of over 50° C. Particularly preferably, reaction mixture is heated to the boiling temperature of the solvent used.

At this temperature the reaction is then carried out over a period of 3 to 9 hours, preferably 4-7, preferably about 6 hours.

Once the reaction is complete, methyl acetate is added and the resulting solution is filtered. The filtrate is heated to 50° C. and acidified, preferably with an inorganic acid, particularly preferably with hydrochloric acid, and after a period of about 10 minutes to 12 hours, preferably 20 minutes to 6 hours, particularly preferably 30 minutes to 3 hours the product is filtered off. During the addition of the acid, inoculation may be carried out with crystals of the compound 1c, for example after the addition of about 5% of the acid used.

The cleaving of the protective group PG from compounds of formula 1c is preferably carried out by hydrogenation in a suitable solvent. Suitable solvents include organic solvents, preferably organic, polar solvents, the particularly preferred solvents being selected from among tetrahydrofuran, various C₃₋₈-esters and C₁₋₈-alcohols. It is preferable according to the invention to use tetrahydrofuran, ethanol and methanol as solvent, of which ethanol and methanol are of particular importance.

For the hydrogenation in the process according to the invention it is preferable to use catalysts in the presence of hydrogen. Preferred catalysts are suitable transition metal catalysts, preferably heterogeneous transition metal catalysts, most preferably palladium-containing catalysts, particularly a mixture of palladium and charcoal.

The hydrogenation is preferably carried out in the presence of an excess of hydrogen. This is provided according to the invention by a hydrogen pressure of 1 bar to 10 bar, preferably between 2 and 7 bar, particularly preferably between 2.5 and 4.5 bar.

Preferably the hydrogenation is carried out at 60° C. After the reaction has ended the catalyst is eliminated, preferably by filtration.

Then the solution is diluted with isopropyl alcohol (IPA) and the product is recrystallised. Preferably the solution is partially evaporated down and the product is crystallised out by cooling the solution.

In a preferred compound according to the invention the compound of formula 1a is prepared by reacting a compound of formula 2a,

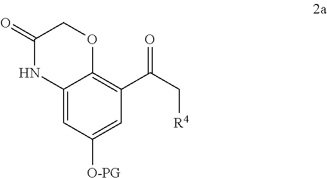

wherein PG has the meaning given in claim 1 and R⁴ denotes halogen, preferably bromine or chlorine.

In the process according to the invention a compound of formula 2a is reacted with (−)-DIP-chloride (diisopinocampheylchloroboran) in a suitable solvent. Preferably, organic solvents are considered as suitable solvents. Preferred solvents are selected from among diethyl ether, tert-butyl-methylether 2-methyltetrahydrofuran, tetrahydrofuran, toluene and dioxane. It is particularly preferred according to the invention to use tert-butyl-methylether, tetrahydrofuran and dioxane as solvent, while particular importance attaches to dioxane and tetrahydrofuran.

The (−)-DIP-chloride may be used in pure form or in the form of a solution, preferably in an inert, organic solvent, particularly preferably an aliphatic solvent, particularly pentane, hexane, heptane or octane, particularly heptane.

The (−)-DIP-chloride is added to the reaction medium at a lower temperature; the temperature is preferably below 0° C., particularly preferably below −10° C., and in particular the addition is carried out at −20 to −40° C.

The (−)-DIP-chloride is added over a period of 10 min to 6 hours, preferably from 30 min to 4 hours, particularly preferably from 1 to 3 hours. In particular, it is added over a period of 70 to 110 min.

Preferably at least stoichiometric amounts of (−)-DIP-chloride are used according to the invention, based on the compound 2a used. If desired the (−)-DIP-chloride may also be used in an excess, for example up to 3 equivalents, preferably up to 2.5 equivalents, particularly preferably about 1.5 to 2.5, particularly about 1.8 equivalents, based on the compound 2a used.

After the addition of the (−)-DIP-chloride the reaction mixture is stirred over a period of 10 min to 4 hours, preferably 30 min to 3 hours, particularly preferably 40 to 80 min, and in particular after the addition has ended the reaction mixture is stirred for a further 50 to 70 min. During this time, the reaction mixture is adjusted to a temperature below 0° C., particularly preferably below −10° C., particularly from −20 to −40° C.

Then, based on the amount of (−)-DIP-chloride used, an at least stoichiometric amount of sodium hydroxide (NaOH), dissolved in water, is added. The NaOH may optionally also be used in an excess, for example up to 3 equivalents, preferably up to 2.5 equivalents, particularly preferably about 1.5 to 2.5, particularly about 1.8 equivalents, based on the amount of DIP-chloride used. Preferably, after the addition of NaOH, a pH of 12 to 14, particularly preferably 12.5 to 13.5, particularly 12.7 to 13.3, is measured in the reaction mixture.

Once the desired pH has been obtained, the reaction mixture is stirred for a period of 10 min to 4 hours, preferably 30 min to 3 hours, particularly preferably 40-80 min, and in particular the reaction mixture is stirred for another 50-70 min. During this time, the reaction mixture is adjusted to a temperature of 0 to 40° C., particularly preferably 10 to 30° C., particularly 15 to 25° C. Then the reaction mixture is adjusted to a pH of 7 to 10, particularly preferably 8 to 9, particularly 8.2 to 8.8, with an acid, preferably an inorganic acid, particularly preferably hydrochloric acid.

Finally the product may be isolated from the reaction mixture by extraction with an organic solvent (preferably ethyl acetate) and) obtained as a solid by precipitation with another suitable organic solvent (preferably methyl-tert-butylether). Before the precipitation the solvent mixture may be partly distilled off.

According to the invention a process is preferred in which the compound of formula 1b is prepared by reacting a compound of formula 2b,

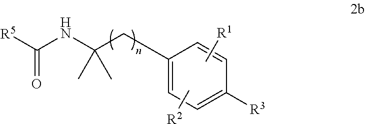

wherein R¹, R², R³ and n are defined as in claims 1 to 5 and
R⁵ denotes C₁₋₆-alkyl, preferably Me.

In the process according to the invention a compound of formula 2b is reacted with sodium hydroxide in a suitable solvent. Examples of suitable solvents include organic solvents; particularly preferred solvents are selected from among ethanol, 2-ethoxyethanol, ethyleneglycol, butoxyethanol, methoxypropanol, propyleneglycol or mixtures thereof. Particularly preferably according to the invention 2-ethoxyethanol or ethyleneglycol or a mixture thereof is used as solvent. Preferably the mixture consists of 2-ethoxyethanol and ethyleneglycol (2:1).

Based on the compound 2b used, preferably at least stoichiometric amounts of the strong base are used according to the invention. The strong base may optionally also be used in excess, for example up to 8 equivalents, preferably up to 6 equivalents, preferably about 2 to 6, particularly preferably 4.5 to 5.5 equivalents, based on the compound 2b used.

The reaction is preferably carried out at elevated temperature, preferably at a temperature of above 100° C., particularly preferably with refluxing of the solvent and over a period of 2-3 hours.

Then for extraction the reaction mixture is diluted with a solvent and water. As the solvent, toluene, xylene, heptane, methylcyclohexane or tert-butyl-methylether, preferably toluene or xylene, are of particular importance. The aqueous phase is eliminated, the organic phase is extracted with water in further purification steps. The water may be made acidic, neutral or alkaline by the use of common additives. Preferably the organic phase is extracted with basic or neutral water. The product is isolated from the organic phase as the HCl salt by the addition of a suitable amount of HCl, at least 60° C., preferably approx. 80° C., with subsequent azeotropic distillation followed by cooling to ambient temperature.

### Terms and Definitions Used

By an “organic solvent” is meant, within the scope of the invention, an organic, low-molecular substance which can dissolve other organic substances by a physical method. To be suitable the prerequisite for the solvent is that neither the dissolving substance nor the dissolved substance should be chemically altered during the dissolving process, i.e. the components of the solution should be recoverable in their original form by physical separation processes such as distillation, crystallisation, sublimation, evaporation or adsorption. For various reasons, not only the pure solvents but also mixtures that combine the dissolving properties may be used. Examples include:
alcohols, preferably methanol, ethanol, propanol, butanol, octanol, cyclohexanol;
glycols, preferably ethyleneglycol, diethyleneglycol;
ethers/glycolethers, preferably diethyl ether, tert-butyl-methylether, dibutylether, anisole, dioxane, tetrahydrofuran, mono-, di-, tri-, polyethyleneglycol ethers;
ketones, preferably acetone, butanone, cyclohexanone;
esters, preferably acetic acid esters, glycolesters;
amides and other nitrogen compounds, preferably dimethylformamide, pyridine, N-methylpyrrolidone, acetonitrile;
sulphur compounds, preferably carbon disulphide, dimethylsulphoxide, sulpholane;
nitro compounds, preferably nitrobenzene;
halogenated hydrocarbons, preferably dichloromethane, chloroform, tetrachloromethane, tri- and tetrachloroethene, 1,2-dichloroethane, chlorofluorocarbons;
aliphatic or alicyclic hydrocarbons, preferably benzines, petroleum ether, cyclohexane, methylcyclohexane, decaline, terpene-L; or
aromatic hydrocarbons, preferably benzene, toluene, o-xylene, m-xylene, p-xylene;
   or corresponding mixtures thereof.

By the term “C₁₋₄-alkyl” (including those which are part of other groups) are meant branched and unbranched alkyl groups with 1 to 4 carbon atoms. Examples include: methyl, ethyl, n-propyl, iso-propyl, n-butyl, iso-butyl, sec-butyl or tert-butyl. In some cases the abbreviations Me, Et, n-Pr, i-Pr, n-Bu, i-Bu, t-Bu, etc. Are also used for the above-mentioned groups. Unless stated otherwise, the definitions propyl and butyl include all the possible isomeric forms of the groups in question. Thus, for example, propyl includes n-propyl and iso-propyl, butyl includes iso-butyl, sec-butyl and tert-butyl etc.

By the term “C₁₋₄-alkylene” (including those which are part of other groups) are meant branched and unbranched alkylene groups with 1 to 4 carbon atoms. Examples include: methylene, ethylene, propylene, 1-methylethylene, butylene, 1-methylpropylene, 1,1-dimethylethylene or 1,2-dimethylethylene. Unless stated otherwise, the definitions propylene and butylene include all the possible isomeric forms of the groups in question with the same number of carbons. Thus, for example, propyl also includes 1-methylethylene and butylene includes 1-methyl propylene, 1,1-dimethylethylene, 1,2-dimethylethylene.

By the term “C₁₋₈-alcohol” are meant branched and unbranched alcohols with 1 to 8 carbon atoms and one or two hydroxy groups. Alcohols with 1 to 4 carbon atoms are preferred. Examples include: methanol, ethanol, n-propanol, iso-propanol, n-butanol, iso-butanol, sec-butanol or tert-butanol. In some cases the abbreviations MeOH, EtOH, n-PrOH, i-PrOH, n-BuOH, i-BuOH, t-BuOH, etc. Are optionally also used for the above-mentioned molecules. Unless stated otherwise, the definitions propanol, butanol, pentanol and hexanol include all the possible isomeric forms of the groups in question. Thus for example propanol includes n-propanol and iso-propanol, butanol includes iso-butanol, sec-butanol and tert-butanol etc.

By the term “C₃₋₈-esters” are meant branched and unbranched esters with a total of 3 to 8 carbon atoms. Esters of acetic acid with 3 to 6 carbon atoms are preferred. Examples include: methyl acetate, ethyl acetate, n-propyl acetate, i-propyl acetate or n-butyl acetate, of which ethyl acetate is preferred.

“Halogen” within the scope of the present invention denotes fluorine, chlorine, bromine or iodine. Unless stated to the contrary, fluorine, chlorine and bromine are regarded as preferred halogens.

“Protective groups” for the purposes of the present invention is a collective term for organic groups with which certain functional groups of a molecule containing a number of active centres can temporarily be protected from attack by reagents so that reactions take place only at the desired (unprotected) sites. The protective groups should be introduced selectively under mild conditions. They must be stable for the duration of the protection under all the conditions of the reactions and purifying procedures which are to be carried out; racemisations and epimerisations must be suppressed. Protective groups should be capable of being cleaved again under mild conditions selectively and ideally in high yields. The choice of a suitable protective group, the reaction conditions (solvent, temperature, duration, etc.), and also the options for removing a protective group are known in the art (e.g. Philip Kocienski, Protecting Groups, 3rd ed. 2004, THIEME, Stuttgart, ISBN: 3131370033). Preferred protective groups are optionally substituted benzyl, diphenylmethyl, trityl, tosyl, mesyl or triflate, of which optionally substituted benzyl is particularly preferred.

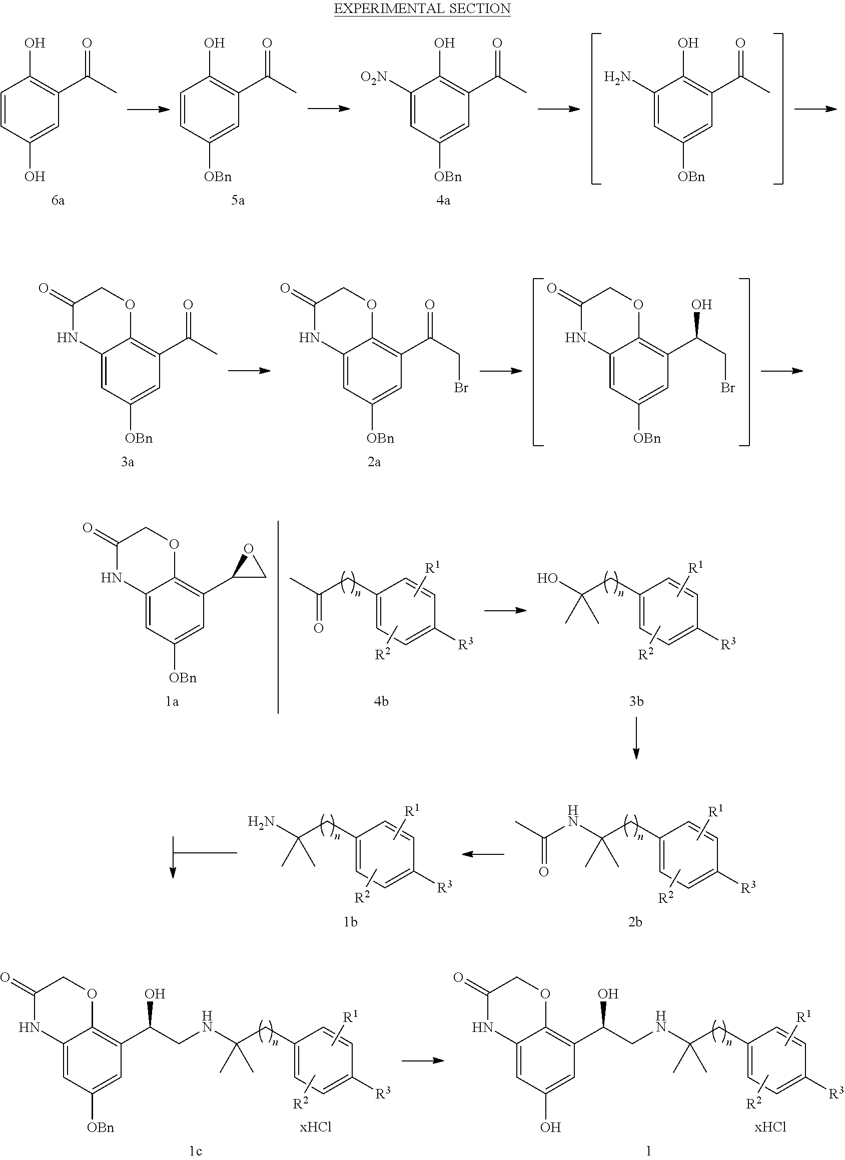

wherein Bn denotes benzyl and
n may denote 1 or 2;
R¹ may denote hydrogen, halogen, C₁₋₄-alkyl or O—C₁₋₄-alkyl;
R² may denote hydrogen, halogen, C₁₋₄-alkyl or O—C₁₋₄-alkyl;
R³ may denote hydrogen, C₁₋₄-alkyl, OH, halogen, O—C₁₋₄-alkyl, O—C₁₋₄-alkylene-COOH, O—C₁₋₄-alkylene-COO—C₁₋₄-alkyl.

8-[(1R)-1-hydroxy-2-[[2-aryl-1,1-dimethyl-ethyl]-amino]ethyl]-6-(phenylmethoxy)-2H-1,4-benzoxazin-3(4H)-one-hydrochloride of formula 1c: 45 mol aryl-1,1-dimethyl-ethylamine hydrochloride 1b are suspended in 12 L water and 60 L toluene are added. 4.3 kg of sodium hydroxide solution (45%) are added with stirring, and the phases are separated. 12 kg (40 mol) 8-(2R)-oxiranyl-6-(phenylmethoxy)-2H-1,4-benzoxazin-3(4H)-one 1a are added to the organic solution and heated to reflux temperature, (during which time approx. 24 L toluene are also distilled off) and stirred for 6 hours at this temperature. Then the mixture is cooled to 55° C., 96 L of methyl acetate are added and at this temperature 3.8 kg (39 mol) hydrochloric acid (30%) are added within 15 minutes. After the addition of approx. 5% of the hydrochloric acid the mixture is inoculated with crystals of 1c. The resulting suspension is cooled to 20° C. and stirred for another 2 hours. The product is centrifuged, washed with 24 L methyl acetate and dried in vacuo at 50° C.

Yield (1c): 80-90%, enantiomeric purity according to HPLC: 95.0-99.5%.

6-hydroxy-8-[(1R)-1-hydroxy-2-[[2-aryl-1,1-dimethyl-ethyl]-amino]ethyl]-2H-1,4-benzoxazin-3(4H)-one-hydrochloride of formula 1: 19.49 mol 8-[(1R)-1-hydroxy-2-[[2-(4-methoxyphenyl)-1,1-dimethyl-ethyl]-amino]ethyl]-6-(phenylmethoxy)-2H-1,4-benzoxazin-3(4H)-one-hydrochloride of formula 1c are placed in the hydrogenating reactor and suspended with 50 L methanol. 125 g palladium on charcoal 10% (50% water) are suspended in 20 L methanol and placed in the hydrogenating reactor. The mixture is hydrogenated at an internal temperature of 60° C. and at 3 bar of hydrogen pressure until no further uptake of hydrogen can be detected (approx. 1.5 hours). The catalyst is filtered off and rinsed with 20 L methanol. 165 L of i-propanol are metered in, the mixture is heated to 50° C. and 180 L are distilled off under a weak vacuum. When no crystal formation takes place the distillation residue is inoculated. Then within 1 hour it is cooled to 0° C. and then stirred for 1 hour at 0° C., suction filtered and washed with 30 L cold i-propanol and dried in vacuo at 45° C.

The product (approx. 15.6 mol) is dissolved in 26 litres of methanol. The resulting solution is heated to 50° C., filtered clear, and the pressure filter is rinsed with 6.6 litres of methanol. 53 L i-propanol are metered in, the mixture is inoculated and approx. 50 L are distilled off at 50° C. under a weak vacuum. Then within 1 hour the mixture is cooled to 0° C. and then stirred for 1 h at 0° C., suction filtered and washed with 30 L cold i-propanol and dried in vacuo at 45° C. Yield (1): 65-80%.

1-[2-hydroxy-5-(phenylmethoxy)-phenyl]-ethanone: 20 kg (131.4 mol) 2-acetyl-hydroquinone 6a are dissolved in 150 L methylisobutylketone and combined with 19.98 kg (144.6 mol) potassium carbonate. At 60° C., 22.48 kg (131.5 mol) benzyl bromide are added. The reaction mixture is stirred for 20 hours at 60° C. The reaction mixture is cooled to 25° C. and the solid is filtered off. The filtrate is washed twice with a solution of 0.96 kg (11.8 mol) sodium hydroxide solution (50%) and 60 L water at 25° C. The methylisobutylketone is substantially distilled off in vacuo, and the residue is dissolved in 80 L methanol at 60° C. The solution is cooled to 0° C. and stirred for 1 hour at this temperature to complete the crystallisation.

Yield (5a): 24.07 kg (75.6%), chemical purity according to HPLC: 99.2%.

1-[2-hydroxy-3-nitro-5-(phenylmethoxy)-phenyl]-ethanone: 10.00 kg (41.27 mol) of 1-[2-hydroxy-5-(phenylmethoxy)-phenyl]-ethanone 5a are dissolved in 50 L acetic acid. 4.40 kg (45.40 mol) of 65% nitric acid are metered into this solution at 15 to 20° C. The feed vessel is rinsed with 4 L acetic acid. The reaction mixture is stirred for a further 1 hour. After inoculation it is combined with 50 L water. The suspension obtained is stirred for 1 hour at 10° C. to complete the crystallisation. The product is centrifuged and dried at 50° C.

Yield (4a): 10.34 kg (87.2%), chemical purity according to HPLC: 99.0%.

8-acetyl-6-(phenylmethoxy)-2H-1,4-benzoxazin-3(4H)-one: 15.00 kg (52.22 mol) 1-[2-hydroxy-3-nitro-5-(phenylmethoxy)-phenyl]-ethanone 4a, 0.165 kg platinum(IV)oxide and 45 L 2-methyltetrahydrofuran are hydrogenated at 3 bar hydrogen pressure and at an internal temperature of 25° C. until no further uptake of hydrogen can be detected. The catalyst is filtered off and washed with 20 L of 2-methyltetrahydrofuran. 23.09 kg (167.09 mol) potassium carbonate are placed in another reactor, and the reaction mixture from the first reactor is added. The mixture is rinsed with 22 L 2-methyltetrahydrofuran. Then within 30 minutes 9.44 kg (83.55 mol) chloroacetyl chloride are metered into the suspension. After 2.5 hours reaction time at 65° C., 101 L water are added. The aqueous phase is separated off at 55° C. Then 34 L of 2-methyltetrahydrofuran are distilled off from the organic phase in vacuo. After heating to reflux temperature, 180 L methylcyclohexane are metered in within 30 minutes while refluxing. The suspension obtained is cooled to 20° C. and stirred for a further 1 hour at this temperature to complete the crystallisation. Then the precipitate is centrifuged off, washed with 113 L methylcyclohexane and dried at 50° C.

Yield (3a): 12.70 kg (81.8%), chemical purity according to HPLC: 98.4%.

8-(bromoacetyl)-6-(phenylmethoxy)-2H-1,4-benzoxazin-3(4H)-one: 12.00 kg (40.36 mol) 8-acetyl-6-(phenylmethoxy)-2H-1,4-benzoxazin-3(4H)-on 3a are dissolved in 108 L of 1,4-dioxane. Then a solution of 24.33 kg (50.45 mol) tetrabutylammonium tribromide in 48 L of 1,4-dioxane and 12 L of methanol is metered in to the suspension at 20° C. The contents of the reactor are stirred for 2 hours at 20° C. Then 72 L water are added at 20° C. within 15 minutes. After cooling to 3° C. the mixture is stirred for 1 hour, centrifuged and washed with a mixture of 9 L of 1,4-dioxane and 4.5 L of water. Then the resulting mixture is washed with 60 L water and dried in vacuo at 50° C.

Yield (2a): 11.29 kg (74.4%), chemical purity according to HPLC: 98.0%.

8-(2R)-oxiranyl-6-(phenylmethoxy)-2H-1,4-benzoxazin-3(4H)-one: 20.00 kg (31.90 mol) 8-(bromoacetyl)-6-(phenylmethoxy)-2H-1,4-benzoxazin-3(4H)-one 2a are dissolved in 200 L tetrahydrofuran and cooled to −30° C. 50.3 kg (70.18 mol) (−)-DIP-chloride in 65% heptane are metered in within 1 hour. The reaction mixture is stirred for another 2 hours and heated to 0° C. At this temperature 18.9 kg (143.54 mol) sodium hydroxide solution (50%), mixed with 40 L water, are metered in. Then the feed vessel is rinsed with 10 L water. The pH at the end of the addition should be 13-13.5. The mixture is heated to 20° C. and stirred for another 1 hour. A mixture of 7.8 kg (80 mol) industrial-grade hydrochloric acid (37%) and 80 L water is metered in until a pH of 8.5 is obtained. After the addition of 100 L ethyl acetate the mixture is heated to 45° C. After phase separation, some of the solvent (approx. 280 L) is distilled off from the organic phase, the residue is combined with 160 L tert-butyl-methylether, cooled to 0° C. and stirred for a further 1 hour. The product is isolated, washed with tert-butylmethylether and dried in vacuo at 50° C. Yield (1a): 13.96 kg (87.0%), enantiomeric purity according to HPLC: 98.3%.

Compounds of formula 3b: 24.68 kg (72.6 mol) methylmagnesium chloride (22% solution in THF) are dissolved in 35 L toluene and cooled to 16° C. At 16-22° C. a solution of 60.9 mol arylacetone of formula 4b and 10 L toluene is metered in and the mixture is stirred for 1 hour at 22° C. The reaction solution is metered into a mixture of 45 L water and 5.22 kg (51.1 mol) sulphuric acid at a temperature of 2-17° C. The two-phase mixture is stirred, and the aqueous phase is separated off. The organic phase is washed with a solution of 1.00 kg (11.9 mol) sodium hydrogen carbonate and 11 L water. The solvent is distilled off completely in vacuo. The residue is dissolved in 65.5 L n-heptane. After cooling to 2° C. the reaction mixture is stirred for 3 hours at this temperature. Then the product is isolated, washed with 17.5 L n-heptane and dried in vacuo at 25° C.

Yield (3b): 75-80%, chemical purity according to HPLC: 98.9-99.9%.

Compounds of formula 2b: 55.48 mol 1-aryl-2-methyl-propan-2-ol of formula 3b are placed in 6.83 kg (166.44 mol) acetonitrile and 13 L acetic acid and heated to 40° C. 5.66 kg (55.48 mol) sulphuric acid are metered in at 50-55° C. Then the mixture is stirred for 3 hours at 50° C. In a second reactor, 160 L water, 20 L tert-butylmethylether and 21 L methylcyclohexane are cooled to 10° C. The contents of the first reactor are transferred to the second reactor. The pH of the reactor contents is adjusted to 9.5 with approx. 40 litres of ammonia solution (25%). The suspension is cooled to 5° C. and stirred for 1 hour at this temperature. The product is centrifuged off and washed with 30 L water and with a mixture of 7.5 L tert-butylmethylether and 7.5 L methylcyclohexane. The moist product is heated to 75° C. in 25 L ethanol (96%) and at this temperature combined with 30 L water. The solution is stirred for 15 minutes at 85° C., then cooled to 2° C. and stirred for 1 hour at this temperature. The product is isolated, washed with a mixture of 5 L water and 5 L ethanol (96%) and dried.

Yield (2b): 65-71%, chemical purity according to HPLC: 98.6-99.8%.

Compounds of formula 1b: A mixture of 136 mol N-[2-aryl-1,1-dimethyl-ethyl]-acetamide of formula 2b, 27.3 kg NaOH (678 mol), 30 L ethoxyethanol and 15 L ethyleneglycol is heated to 150° C. for 3 hours. After cooling to 50-80° C. the mixture is diluted with 90 L of water and 90 L of toluene. The phases are separated and the organic phase is washed once more with 60 L water. The organic phase is combined with 13.4 kg hydrochloric acid (136 mol). After the distillation of 9.5 L of azeotrope the solution is inoculated at above 80° C. and cooled to ambient temperature over 1 hour. The product is centrifuged, washed with 60 litres of toluene and dried in vacuo at 50° C.

Yield (1b): 85-95%, chemical purity according to HPLC: >99.5%.

In the above mentioned syntheses for the compounds of formulae 3b, 2b and 1b the groups R¹, R² and R³ may have the following meanings, for example:

| | R¹ | R² | R³ |
|---|---|---|---|
| Example 1 | H | H | OMe |
| Example 2 | 2-F | H | F |
| Example 3 | 3-F | 5-F | H |
| Example 4 | H | H | OEt |
| Example 5 | H | H | F |

The R-forms of the following compounds of formula 1 may thus be obtained analogously to the preparation methods described hereinbefore:
6-hydroxy-8-{1-hydroxy-2-[2-(4-methoxy-phenyl)-1,1-dimethyl-ethylamino]-ethyl}-4H-benzo[1,4]oxazin-3-one;
8-{2-[2-(2,4-difluoro-phenyl)-1,1-dimethyl-ethylamino]-1-hydroxy-ethyl}-6-hydroxy-4H-benzo[1,4]oxazin-3-one;
8-{2-[2-(3,5-difluoro-phenyl)-1,1-dimethyl-ethylamino]-1-hydroxy-ethyl}-6-hydroxy-4H-benzo[1,4]oxazin-3-one;
8-{2-[2-(4-ethoxy-phenyl)-1,1-dimethyl-ethylamino]-1-hydroxy-ethyl}-6-hydroxy-4H-benzo[1,4]oxazin-3-one;
8-{2-[2-(4-fluoro-phenyl)-1,1-dimethyl-ethylamino]-1-hydroxy-ethyl}-6-hydroxy-4H-benzo[1,4]oxazin-3-one.

1. Process for preparing compounds of formula 1, 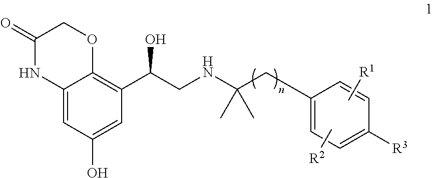
wherein
n denotes 1 or 2;
R¹ denotes hydrogen, halogen, C₁₋₄-alkyl or O—C₁₋₄-alkyl;
R² denotes hydrogen, halogen, C₁₋₄-alkyl or O—C₁₋₄-alkyl;
R³ denotes hydrogen, C₁₋₄-alkyl, OH, halogen, O—C₁₋₄-alkyl, O—C₁₋₄-alkylene-COOH, O—C₁₋₄-alkylene-COO—C₁₋₄-alkyl;
characterised in that a compound of formula 1a, 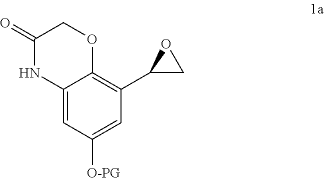
wherein PG denotes a protective group, is reacted with a compound of formula 1b, 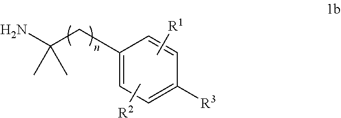
wherein R¹, R², R³ and n have the meanings given above, in toluene, over a period of 3-9 hours, to form a compound of formula 1c, 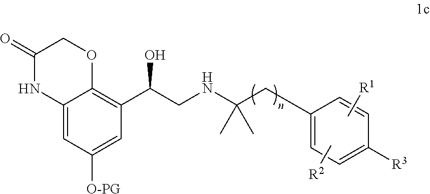
wherein R¹, R², R³, n and PG have the meanings given above, and the compound of formula 1 is obtained therefrom by cleaving the protective group PG.

2. Process for preparing compounds of formula 1 according to claim 1, wherein
n denotes 1 or 2;
R¹ denotes hydrogen, halogen or C₁₋₄-alkyl;
R² denotes hydrogen, halogen or C₁₋₄-alkyl;
R³ denotes hydrogen, C₁₋₄-alkyl, OH, halogen, O—C₁₋₄-alkyl, O—C₁₋₄-alkylene-COOH or O—C₁₋₄-alkylene-COO—C₁₋₄-alkyl.

3. Process for preparing compounds of formula 1 according to claim 1, wherein
n denotes 1 or 2;
R¹ denotes hydrogen, fluorine, chlorine, methyl or ethyl;
R² denotes hydrogen, fluorine, chlorine, methyl or ethyl;
R³ denotes hydrogen, C₁₋₄-alkyl, OH, fluorine, chlorine, bromine, O—C₁₋₄-alkyl, O—C₁₋₄-alkylene-COOH, O—C₁₋₄-alkylene-COO—C₁₋₄-alkyl.

4. Process for preparing compounds of formula 1 according to claim 1, wherein
n denotes 1 or 2;
R¹ denotes hydrogen, methyl or ethyl;
R² denotes hydrogen, methyl or ethyl;
R³ denotes hydrogen, methyl, ethyl, OH, methoxy, ethoxy, O—CH₂—COOH, O—CH₂—COO-methyl or O—CH₂—COO-ethyl.

5. Process for preparing compounds of formula 1 according to claim 1, wherein
n denotes 1 or 2;
R¹ denotes hydrogen or methyl;
R² denotes hydrogen or methyl;
R³ denotes hydrogen, methyl, OH, methoxy, O—CH₂—COOH or O—CH₂—COO-ethyl.

6. Process according to claim 1, wherein the compound of formula 1a is prepared by reacting a compound of formula 2a, 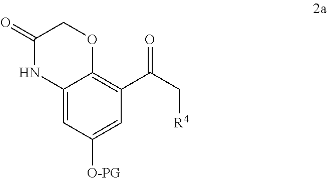
wherein PG has the meaning given in claim 1 and R⁴ denotes halogen, in the presence of a diisopinocampheylchloroborane/heptane solution.

7. Process according to claim 1, wherein the compound of formula 1b is prepared by reacting a compound of formula 2b, 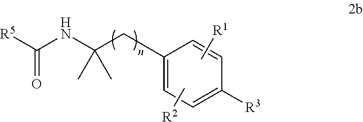
wherein R¹, R², R³ and n have the meanings given in claim 1 and
R⁵ denotes C₁₋₆-alkyl;
and is isolated as the HCl salt.

8. Process for preparing compounds of formula 1b, 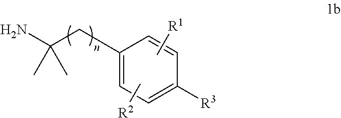
wherein R¹, R², R³ and n have the meanings given in claim 1, characterised in that a compound of formula 2b, 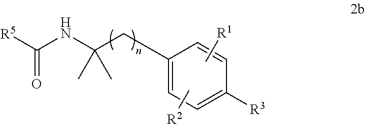
wherein R¹, R², R³ and n have the meanings given in claim 1 and
R⁵ denotes Me;
is reacted with sodium hydroxide and is isolated as the HCl salt.
